(19) United States
(12) Patent Application Publication
(10) Pub. No.: US 2019/0320976 A1
(43) Pub. Date: Oct. 24, 2019

(54) METABOLIC MONITORING SYSTEM

(71) Applicant: Zense-Life Inc., Carlsbad, CA (US)

(72) Inventors: Mitchell Steven Roslin, Amonk, NY (US); Robert James Boock, Carlsbad, CA (US); Eli Reihman, San Diego, CA (US); Leif Bowman, San Diego, CA (US); Todd Noboru Haseyama, Encinitas, CA (US); Katherine Yerre Koehler, Solana Beach, CA (US)

(73) Assignee: Zense-Life Inc., Carlsbad, CA (US)

(21) Appl. No.: 16/386,021

(22) Filed: Apr. 16, 2019

Related U.S. Application Data
(60) Provisional application No. 62/659,537, filed on Apr. 18, 2018.

(51) Int. Cl. A61B 5/00; H04W 4/38; A61B 5/145; G06K 9/00; G06F 3/16

(52) U.S. Cl. CPC A61B 5/4866; H04W 4/38; A61B 5/14532; A61B 5/742; A61B 5/0002; G06K 9/00201; G06F 3/167; A61B 5/486

(57) ABSTRACT

A method for metabolic monitoring includes a processor receiving glucose data associated with an individual from a metabolic sensor and food intake information associated with the individual. The processor calculates a plurality of global metrics. Each global metric is based on a glucose variability, a glucose load, and a post-prandial peak. The glucose variability is calculated from the glucose data associated with the individual. The processor determines an individualized metric by correlating the food intake information associated with the individual to the plurality of global metrics, and recommends a behavior modification based on the individualized metric.

### RELATED APPLICATIONS

This application claims priority to U.S. Provisional Patent Application No. 62/659,537 filed on Apr. 18, 2018 and entitled “Metabolic Monitoring System,” which is hereby incorporated by reference in full.

### BACKGROUND

Monitoring glucose levels is critical for diabetes patients. Continuous glucose monitoring (CGM) sensors are a type of device in which fluid is sampled from just under the skin multiple times a day. CGM devices typically involve a small housing in which the electronics are located and which is adhered to the patient's skin to be worn for a period of time. A CGM sensor, which is often electrochemical, is delivered subcutaneously by a small needle within the device.

Electrochemical glucose sensors operate by using electrodes which detect an amperometric signal caused by oxidation of enzymes during conversion of glucose to gluconolactone. The amperometric signal can then be correlated to a glucose concentration. Two-electrode (also referred to as two-pole) designs use a working electrode and a reference electrode, where the reference electrode provides a reference against which the working electrode is compared. Three-electrode (or three-pole) designs have a working electrode, a reference electrode and a counter electrode. The counter electrode replenishes ionic loss at the reference electrode and is part of the ionic circuit.

Glucose readings taken by the sensor can be tracked and analyzed by a monitoring device, such as by scanning the sensor with a customized receiver or by transmitting signals to a smartphone or other device that has a specific software application. Software features that have been included in CGM systems include viewing glucose levels over time, indicating glucose trends, and alerting the patient of high and low glucose levels.

### SUMMARY

In some embodiments, a method for metabolic monitoring includes a processor receiving glucose data associated with an individual from a metabolic sensor and food intake information associated with the individual. The processor calculates a plurality of global metrics. Each global metric is based on a glucose variability, a glucose load, and a post-prandial peak. The glucose variability is calculated from the glucose data associated with the individual. The processor determines an individualized metric by correlating the food intake information associated with the individual to the plurality of global metrics, and recommends a behavior modification based on the individualized metric.

In some embodiments, a metabolic monitoring system includes a metabolic sensor configured to measure glucose data associated with an individual and a processor configured to receive glucose data associated with the individual from the metabolic sensor. Food intake information associated with the individual is received. A plurality of global metrics are calculated. Each global metric is based on a glucose variability, a glucose load, and a post-prandial peak. The glucose variability is calculated from the glucose data associated with the individual. An individualized metric is determined by correlating the food intake information associated with the individual to the plurality of global metric. A behavior modification is recommended based on the individualized metric.

### BRIEF DESCRIPTION OF THE DRAWINGS

FIG. 1 shows a graph of post-prandial glucose response for various individuals in a prior art study.

FIG. 2 is a comparison of glucose variability values calculated using various methods.

FIGS. 3A-3B and FIG. 4 are graphs of glucose parameters over time.

FIG. 5A is a schematic of a metabolic monitoring system, in accordance with some embodiments.

FIG. 5B is a schematic of the server of FIG. 5A, in accordance with some embodiments.

FIGS. 6A and 6B show flowcharts of methods for monitoring metabolic activity, in accordance with some embodiments.

FIG. 7 is a tree diagram showing inputs that can be used in the present methods, in accordance with some embodiments.

FIGS. 8 and 9 show examples of user interfaces, in accordance with some embodiments.

FIGS. 10A and 10B are embodiments of a user interface for the display of data, in accordance with some embodiments.

FIGS. 11A-11C illustrate user interfaces for the display of data, in accordance with some embodiments.

FIGS. 12A-12C depict embodiments of a user interface to communicate data, in accordance with some embodiments.

FIGS. 13A-13C depict other embodiments of a user interface to communicate data, in accordance with some embodiments.

FIG. 14A shows an embodiment of a secondary user interface display screen to display data, in accordance with some embodiments.

FIG. 14B are examples of simple visuals of suggested foods, drinks or medications, in accordance with some embodiments.

FIG. 15A shows the user interface with a dropdown menu to further communicate data, in accordance with some embodiments.

FIG. 15B is a secondary user interface display screen to display data, in accordance with some embodiments.

FIG. 16A is a user interface for the display of data, in accordance with some embodiments.

FIGS. 16B and 16C depict embodiments of example software applications in communication with the system.

FIG. 17 is a user interface for the display of data, in accordance with some embodiments.

FIG. 18 is a user interface for the display of data, in accordance with some embodiments.

### DETAILED DESCRIPTION

The present embodiments uniquely use direct real-time metabolic data to encourage a user to change or modify behavior related to eating, exercise and subsequent weight loss. Methods and systems are disclosed in which continuous glucose monitoring is used to provide real-time feedback on the impact of eating various foods on post-prandial (post-meal) glucose levels in an individual. A goal of the present methods and systems is to encourage patients to lower the amplitude and number of glucose spikes following eating. While individuals eat and drink according to their likes and dislikes and until a feeling of satiety, the impact of the food and drinks on their body chemistry is unknown. The higher the glucose spike the higher the production of insulin, which leads to fat formation, a crescendo and decrescendo in glucose values, and a greater likelihood of eating more frequently. Each individual reacts to different foods in a unique manner. In the present embodiments, providing information that correlates food intake to glucose metrics can dramatically alter food choices and improve health. Furthermore, weight, exercise, and stress can change these reactions, requiring frequent recalibration. The present embodiments can account for the fact that the body is always changing.

Embodiments disclose a sensor-based system for weight loss, treatment of insulin resistance and its related diseases such as polycystic ovary syndrome (PCOS), non-alcoholic steatohepatitis (NASH), non-alcoholic fatty liver disease (NAFLD), and reducing the likelihood of cancer recurrence. These diseases are related to excess glucose in the body. The present systems include a monitoring system with feedback and professional advice via calculated metrics to help treat the person's obesity or weight management regime, and then creating actionable goals that are communicated on a software application for the patient to utilize and modify their behavior.

Although embodiments shall be described in terms of providing weight loss recommendations for a user, the concepts can be applied to providing other user-derived behavior modifications or correlations. For example, the present methods and systems can recommend behavior modifications such as training programs for athletes, or health management recommendations in relation to a medical condition of a user.

The system includes a software application and a sensor/transceiver that wirelessly communicates to a device which can be, for example, a smartphone, tablet, smart watch, or the like. In some embodiments, all the information is sent to a cloud-based server for analysis. In other embodiments, the information and processing of the data can be performed on the device or by an electronics unit connected to the metabolic sensor. In further embodiments, the sensor/transceiver can be sent to a non-mobile device, such as a desktop computer or a kiosk that may be located in a facility such as a doctor's office or hospital. Analytics, which can be cloud-based or can be included in the software application of the device, create personalized advice with personalized metrics based directly on the individual's data and can be distributed back to the patient and/or sent to a physician, dietician, trainer or family member.

The metrics are based upon glucose variability and total glucose load, uniquely combining multiple glucose indicators to form a global metric that serves as a metabolic index. The glucose information is sent to artificial intelligence (AI) programs, which may be cloud-based, to correlate the global metrics with food intake. The metrics and food intake information may also be correlated with numerous other measures such as heart rate (HR), location, activity, etc., to provide a more complete picture of the person's individual metabolic profile.

Many embodiments are included for displaying data on a user interface device. The data may be information such as the mean glucose level or another metric that is calculated or obtained from the sensor of the continuous glucose monitoring. The recommended behavior modifications may also be displayed which includes eating a food, drinking a beverage, taking medication, or performing an exercise. These are specific activities and quantities based on the historical food intake information received and historical metabolic index calculations for the specific individual. In some embodiments, the processor is part of a device such as a mobile phone, having a lock screen, home screen or wallpaper feature. The lock screen, home screen or wallpaper may be modified based on the glucose data associated with an individual from a metabolic sensor. In other words, the lock screen, home screen and/or wallpaper of the device may be modified based on the glucose data from the sensor.

Clinical evidence data from various trials and studies show a correlation between weight loss and glucose variation, a link between obesity and glucose variation, and a correlation between food intake and glucose variation.

Evidence of a correlation between weight loss and glucose variation was first shown in the FLAT-SUGAR trial. This trial compared insulin to GLP-1 Agonist (a drug that controls glucose variation) in the hope of improving A1c levels (a measure of glycosolated hemoglobin linked to long-term health risks). The study showed no marked change in A1c even when marked reductions in glucose variations were shown. However, an unexpected observation from the study was a dramatic and sustained weight loss (4.5 kg or 10.6 lbs over 26 weeks) in the group with dramatically reduced glucose variation.

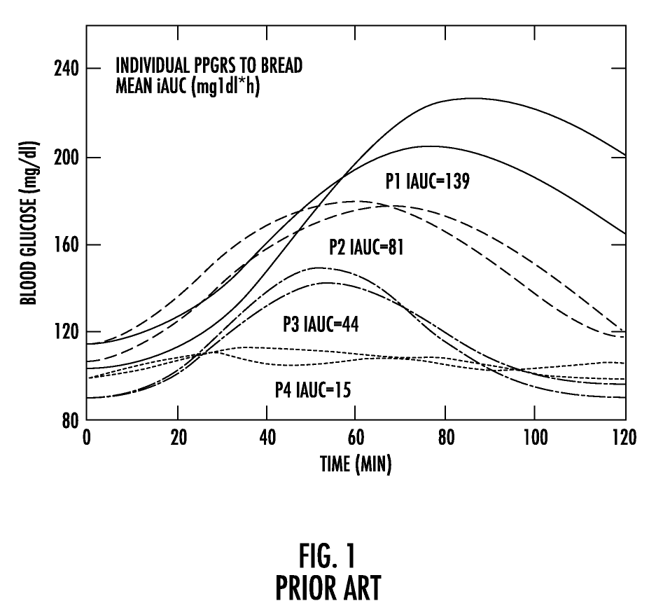

Evidence of a link between obesity and glucose variation was demonstrated in a paper by Salkind et al. This study showed that higher glucose variability exists in overweight, pre-diabetic and obese patients compared to non-diabetic adult controls. It remains to be demonstrated whether this is a cause or an effect. A study by Trico et al. showed that by simple changes in the order of food intake, glucose response can be altered. In other words, glucose variation can be reduced through changing the food intake sequence. This study, which focused on post-prandial (meal) peaks in glucose, demonstrates that simple advice can be used to modify glucose response to foods. A further consideration in correlating food intake to glucose response is that every individual responds to food differently. For example, FIG. 1 is a graph of post-prandial glucose response (PPGR) of the blood glucose in mg/dl over time showing the insulin area under the curve (IAUC). The graph shows that four people (P1, P2, P3, P4) given the exact same bread to eat had wide variations in their glucose response. For example, person 1 (P1) had an IAUC of 139 while person 4 (P4) had an IAUC of 15. It can be seen from FIG. 1 that weight loss products must be tailored to the individual and their physiologic response to be most effective as opposed to a one-size-fits-all approach.

The present embodiments personalize a program to control blood glucose variations and overall glucose load for a consumer by using global metrics that are a unique combination of multiple glucose indicators. The global metrics are essentially metabolic indices and are utilized to determine an individualized metric, where the individualized metric is customized for that particular consumer. By controlling blood glucose levels, the consumer can improve their health which is beneficial for managing diseases such as diabetes and for weight loss.

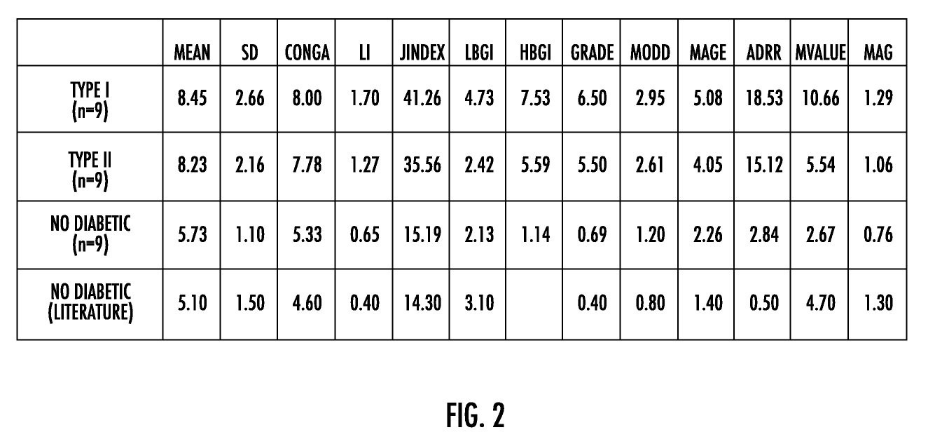

To derive the unique global metrics of the present disclosure, a pre-existing data set was first examined to calculate concepts and to determine whether continuous calculation of variation would provide new data or insights to use in a weight loss product. FIG. 2 shows a comparison of glucose variation across normal, Type I diabetic and Type II diabetics (normally associated with being overweight) calculated by a number of methods. The glucose variation (GV) data shown in FIG. 2 were 7-day averages calculated using EasyGV online software. The calculation types include mean, standard deviation (SD), continuous overall net glycemic action (CONGA), lability index (LI), J-index (J=0.324*(MBG+SD)²), low blood glucose index (LBGI), high blood glucose index (HBGI), glycemic risk assessment diabetes equation (GRADE), mean of daily differences (MODD), mean amplitude of glycemic excursions (MAGE), average daily risk ratio (ADRR), M-value of Schlichtkrull, and mean absolute glucose (MAG). The 7-day global calculations of these populations were in line with those reported in the literature, showing this data set to be representative. The calculations by the various GV methods show that a significant difference between diabetic and non-diabetic populations was observed.

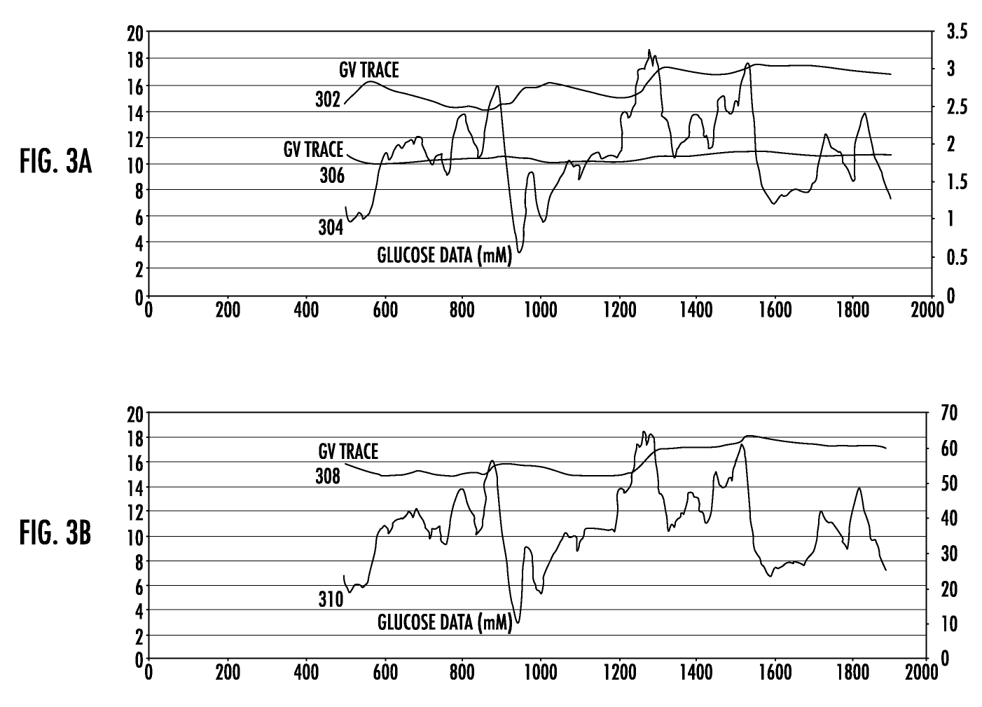

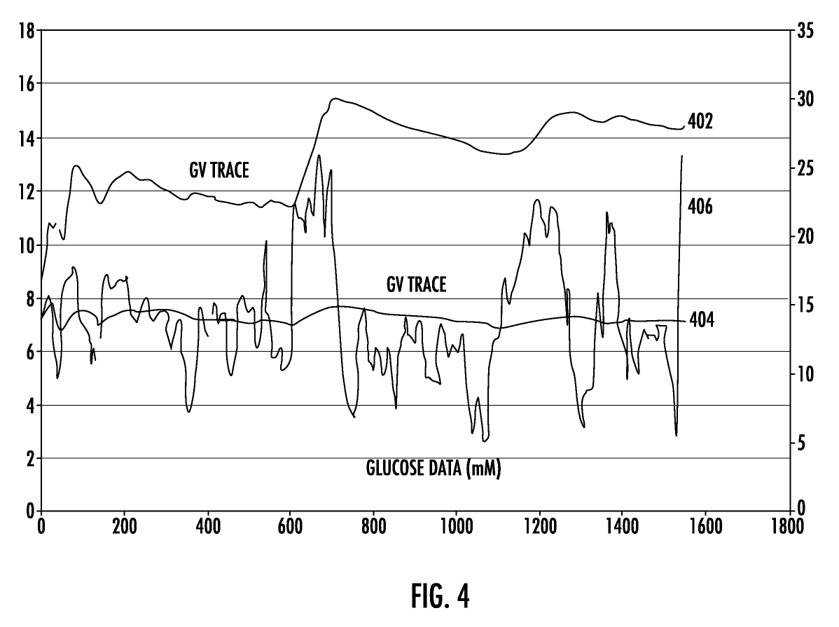

Next, a new concept of tracking glucose variability in real-time was investigated. FIGS. 3A-3B show continuous glucose variation calculations overlaying continuous glucose data for a sample Type I patient. FIG. 3A shows real-time glucose variation values calculated by SD (GV Trace 302), compared to glucose data values, line 304. FIG. 3A also shows the average glucose or Glucose Load, labeled as the mean (line 306). FIG. 3B is similar to FIG. 3A but uses the J-index method for the GV Trace, line 308 compared to glucose data values, line 310. FIG. 4 is a graph for another Type I patient, showing a J-index GV Trace as line 402, a mean GV trace as line 404, and glucose data values as line 406. These graphs demonstrate that continuous calculation of glucose variation is distinct from continuous glucose values and thus offers more and different data to be used in making a weight loss product. For example, the slopes, locations of peaks and valleys, and trends for glucose variability are different from those for the glucose values. As a specific example, the glucose variation (J-index line 402) in FIG. 4 continues to climb over the duration, indicating the blood glucose variations and overall glucose load of the person is out of control.

The present embodiments uniquely use the concept of monitoring real-time glucose variation to formulate metrics for a weight loss program, where the metrics are personalized for an individual's specific characteristics. Terminology used in calculating the metrics is listed below:
GV=glucose variability (calculated by any number of formulas, such as those known in the art);
GL=glucose load or the average glucose value over a time period, such as 1 day;
PPP=post-prandial (meal) peak;
Global Metric=weight loss metric to be displayed to the patient.

The global metrics are unique indices of the present embodiments that use weighting factors to combine GV, GL and PPP. The weighting factors are tailored for the data of an individual person, such as by curve-fitting the data for the individual.

Listed below are example formulas for the metrics, where A, B and C are weighting factors that can be either constants or derived functions. Other formulas for global metrics may be used, and one or more of these derived metrics may be shown on-screen in the software application used by the patient.
Global Metric 1=A*GV+B*GL+C*PPP
Global Metric 2=C*PPP/(A*GV+B*GL)
Global Metric 3=A*GV/B*GL+PPP
Global Metric 4=(A*GV+C*PPP)/B*GL
Global Metric 5=C*PPP/(A*GV+B*GL)

The derived functions for weighting factors A, B and C can be, for example, a polynomial function, exponential function, logarithmic function or power-law function. In some embodiments, a rate of change may be used as part of the functions, such as a rate of change of metabolic sensed values where rapid rise or rapid decrease of these values correspond to certain behaviors such as eating or exercise. For example, during rapid rates of change these weighting factors may increase how portions of the index (global metric) may be weighted, such as the post prandial peak value. The five examples of global metric calculations above use different additive and multiplicative combinations.

Each variable in the overall (global) metric is likely to be weighted differently for each individual. For example, GV is known to rise as a person goes from normal weight to overweight to obese, and higher values of GV are known to be correlated to those who are overweight. Thus, for higher weight individuals, the weighting factor “A” for GV in the global metric of the present embodiments may be higher than for people with lower or normal weight. in another example, the PPP is often more muted in the morbidly obese population than people in the overweight category, and thus the weighting factor “C” of PPP in the global metric of the present embodiments may be lower in value for morbidly obese patients compared to overweight individuals. In yet another example, the GL may be more correlative for weight gain or loss in the normal population rather than in the overweight or obese populations. Consequently, GL may have a higher weighting factor “B” for individuals in lower weight categories. Note that these examples describe general trends, which may not apply to every case since the actual weighting factors for each situation is highly individualized. Furthermore, although these examples show how a person's weight category can be used to affect the derived functions or rate of change for the weighting ors, other aspects may be used to tailor the weighting factors.

The rate of change can also differ widely in different cohorts. For example, for PPP a fast rate of change of may potentially result in the PPP being more correlated to weight gain even though the PPP value is low. The rate of decline from a PPP may be especially relevant to long term weight gain with a slow decline more likely to be correlated to weight gain.

The present glucose variation monitoring and weight loss systems and methods integrate continuous glucose monitoring with, in some embodiments, image and auditory recognition software to provide information in a single displayed screen that predicts an individual's post-prandial glucose and guide food selection. The system receives meal inputs from the user to input what is being eaten. Then using analytics, which may be cloud-based, the system generates a series of parameters for the meal. Based on the meal parameters and the CGM measurements, the system calculates and displays actionable targets for the patient that are communicated back to the patient and displayed as behavior modification recommendations.

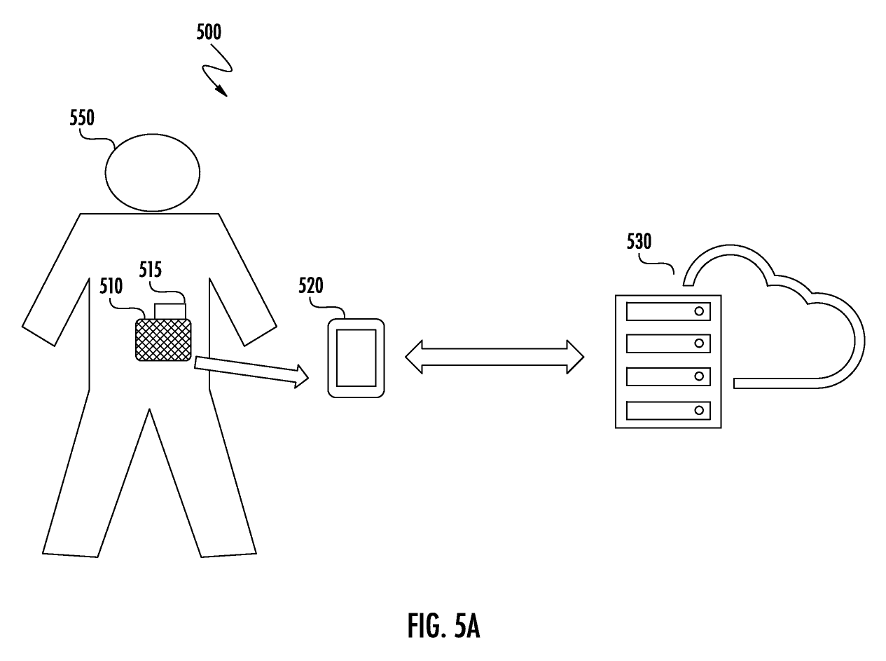

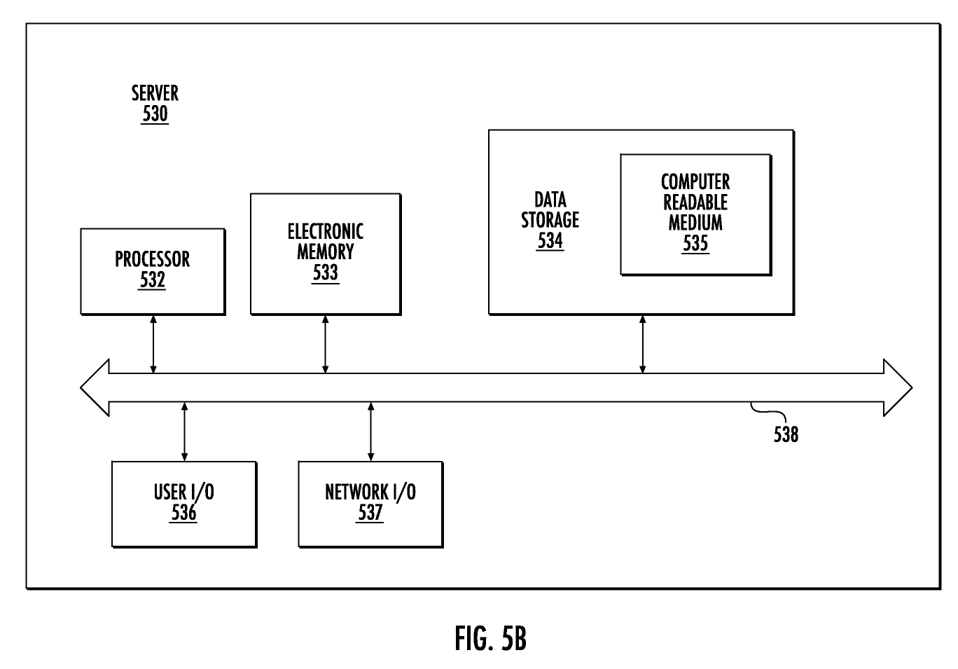

FIG. 5A is a schematic of a metabolic monitoring system 500 which includes a metabolic sensor 510, an electronic device 520 and a server 530, which is depicted as being cloud-based. The metabolic sensor 510 shall be described as a CGM sensor but can also measure other metabolic characteristics such as ketones or fatty acids. For example, the metabolic sensor 510 can represent the use of multiple types of sensors or can represent a single sensor that is configured to measure multiple types of substances. The CGM sensor 510 and typically, a wearable patch, may be applied to the patient 550 by a CGM applicator, where the sensor 510 takes glucose and/or other metabolic readings from under the surface of the patient's skin. The CGM sensor 510 may be connected to an electronics unit 515 in the wearable patch and the electronics unit 515 is configured to transmit glucose data readings wirelessly to an electronic device 520, which may be, for example, a mobile device such as a smartphone, a tablet, or smart watch, or a laptop computer. In some embodiments, the electronic device 520 is not mobile but may be, for example, a desktop computer or medical equipment configured to receive readings from the sensor 510 via the electronics unit 515.

The device 520 receives food intake information (e.g., food eaten during or between meals) from the patient 550, and the food information and glucose readings are transmitted to the server 530. The transmission may be accomplished through a variety of paths, communication access systems or networks. The networks may be the Internet, a variety of carriers for telephone services, third-party communication service systems, third-party application cloud systems, third-party customer cloud systems, cloud-based broker service systems (e.g., to facilitate integration of different communication services), on-premises enterprise systems, or other potential data communication systems. The server 530 can represent a cloud-based processing system. In other embodiments, the meal and glucose data can be stored and processed on the device 520 itself, such that the server 530 is not required.

FIG. 5B is a simplified schematic diagram showing an embodiment of server 530 (representing any combination of one or more of the servers) for use in the system 500, in accordance with some embodiments. Other embodiments may use other components and combinations of components. For example, the server 530 may represent one or more physical computer devices or servers, such as web servers, rack-mounted computers, network storage devices, desktop computers, laptop/notebook computers, etc., depending on the complexity of the metabolic monitoring system 500. In some embodiments implemented at least partially in a cloud network potentially with data synchronized across multiple geolocations, the server 530 may be referred to as one or more cloud servers. In some embodiments, the functions of the server 530 are enabled in a single computer device. In more complex implementations, some of the functions of the computing system are distributed across multiple computer devices, whether within a single server farm facility or multiple physical locations. In some embodiments, the server 530 functions as a single virtual machine.

In the illustrated embodiment, the server 530 generally includes at least one processor 532, a main electronic memory 533, a data storage 534, a user input/output (I/O) 536, and a network I/O 537, among other components not shown for simplicity, connected or coupled together by a data communication subsystem 538. A non-transitory computer readable medium 535 includes instructions that, when executed by the processor 532, cause the processor 532 to perform operations including calculations of global metrics, determining of an individualized metric, and providing behavior modification recommendations as described herein.

In accordance with the description herein, the various components of the system or method generally represent appropriate hardware and software components for providing the described resources and performing the described functions. The hardware generally includes any appropriate number and combination of computing devices, network communication devices, and peripheral components connected together, including various processors, computer memory (including transitory and non-transitory media), input/output devices, user interface devices, communication adapters, communication channels, etc. The software generally includes any appropriate number and combination of conventional and specially-developed software with computer-readable instructions stored by the computer memory in non-transitory computer-readable or machine-readable media and executed by the various processors to perform the functions described herein.

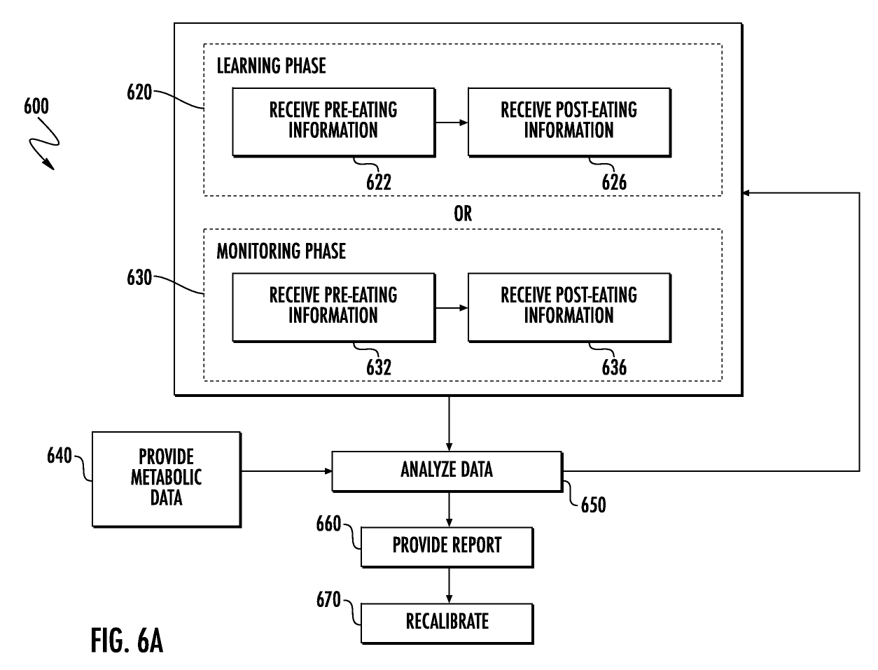

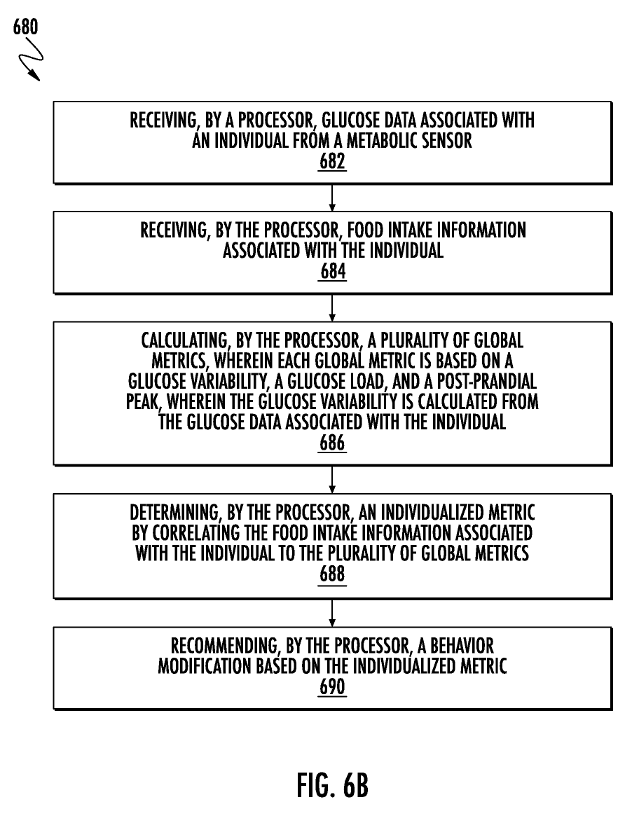

FIG. 6A is a flowchart showing a method of monitoring metabolic activity 600, such as glucose variability, in accordance with some embodiments. The steps of the method 600 may be implemented on a non-transitory machine-readable medium, such as a software application on a computer processor. The method 600 begins with a learning phase 620 in which information pre-eating is received by the system in step 622. In various embodiments, the food intake information can be input by one or more methods such as uploaded images or photographs, audio (e.g., voice) input, video recordings, or typed text on the device or other input system. In some embodiments, the inputs may be by a third-party such as a software application. In this implementation, the user inputs the food intake information into the software application and the data is uploaded to the system. The system can use image recognition and/or voice recognition for identifying the food intake information that is received from the user, such as identifying a food item and an amount of the food item consumed. For example, before eating, step 622 may involve uploading a picture of what is to be consumed as well as receiving a verbal entry about the food. The patient then consumes the food. After eating, the system receives food information in step 626 which can include receiving another picture along with a verbal estimate of the percentage of the total food that was consumed. If there is insufficient information received, the system may prompt the user to enter the missing information. For instance, the system may determine from the post-meal photo that there has been a decline in food present and can request verbal entry of the amounts and/or types of food consumed.

In step 640 metabolic data including glucose data is provided by the metabolic (CGM) sensor. In step 650 the system, in some embodiments, the server or the device, analyzes the data—that is, the food information and glucose readings from the CGM. The food information can include the types of food, amounts, and sequence in which the food items were eaten. Individual metrics may be generated and displayed for the patient and are based on the calculated global metrics described herein. The global metrics are based on glucose variability using formulas that combine GV, GL and PPP using weighting factors depending on each individual. Displayed metrics may also include possible rates of metabolic change.

The system then begins to predict the patient's PPG and whether the meal will be in a high, borderline, or safe zone for the particular patient. These PPG zones may be indicated visually on the display of the device by, for example, red, yellow, and green colors, respectively. Determination of which global metric to use as the individualized metric to display for a patient can be based on factors such as their weight category, the presence of a diabetic condition, or their individual historical trends. For example, the determining of the individualized metric may include learning from the received historical food intake information associated with the patient and historical metabolic index calculations. A behavior modification recommendation is generated by analyzing correlations between the global metric and food intake, where the analysis may use artificial intelligence (AI) in some embodiments.

An example of a behavior modification recommendation based on the metrics is suggesting an order of eating foods in a meal, such as eating protein or fat first to produce lower GV and PPP for a particular person. In another example, an individual may have high glucose responses to certain foods (i.e., carbohydrates), and recommendations can be made by the system to substitute foods that result in a lower response. These substitutions could be alternative types of food items or could be another food within the same food type, based on the individual's own data on how they respond to each type of food eaten. Over time, the system's response database (e.g., data storage 534 of FIG. 5B) of the patient's glucose responses and food intake information grows and more correlations are gained, and better advice on food substitutions becomes available to the user. The individual response to food and food groups is not static and changes over time as the individual loses weight, so constant updating of the response database is performed by the system. In some embodiments, meal and exercise timing can also be correlated to help the individual produce lower metrics (GV and GL in particular) in order to produce weight loss and to sustain weight loss in the individual.

The cycle of steps 620, 640 and 650 then repeats so that the system can learn the patient's typical metabolic responses. Once there is a reasonable match between the predicted and actual results the learning phase is complete. The learning phase can also be used to train the analysis system on voice recognition of audio input of food intake information from the individual.

The patient continues to use the application in a monitoring phase 630 and the system receives pre-eating information in step 632 (e.g., by receiving an uploaded picture of what is eaten), and receives post-eating information in step 636 after the food is eaten. As described in relation to step 620, in some embodiments the system uses a mobile device input (e.g., by smart phone) of food intake via photos and/or voice-driven inputs to obtain caloric estimates. However, receiving food information from other devices is also possible, such as by a desktop computer that can then send the information to a mobile device or to a computer server that has the metabolic readings.

For calculations during the monitoring phase, in step 640 glucose data is again provided to the system by the metabolic (CGM) sensor 510 via the electronics unit 515. In step 650 the system analyzes the food information (e.g., a meal, drink, or snack) and the glucose readings from the CGM to correlate the food intake information to the global metrics. The system can then calculate a prediction of a glucose level zone the patient will be in. If there is a spike in glucose level without food data entry, the system requests entry of the information. The predictions can be performed in real-time, thus providing useful information for the user to monitor their metabolic and alter their behavior immediately as needed. Metabolic sensors continuously measure and track the patient for a desired time period, such as several days (e.g., up to 14 days). The process of analyzing the data in step 650 continues during this time period, using the meal information from monitoring phase 630 and CGM data in step 640.

The analysis during the monitoring phase 630 may continue to use the individualized metric that was determined during the learning phase 620 or may change the individualized metric to adapt to changes in the user's response. Changing the individualized metric may involve adjusting the weighting factors and/or changing which global metric to use for the individualized metric. In some embodiments the behavior in the monitoring phase 630 may be different than in the learning phase 620 due to information regarding meals not being received. In such cases, the system sends reminders to the patient that data is not received and suggests repeat CGMs.

In step 660 a report is generated periodically (daily, for example), that provides information such as the mean glucose level, number of spikes, highest spike, foods that caused spikes, and the like. The displayed information may be generated as an aggregate value (day by day, weekly, etc.) or for each individual meal or activity. This information can be presented visually, such as percentages of meals in red, yellow, or green zones, where the zone categories are based on which global metric is used to serve as the individualized metric. The reports may include a daily predicted mean glucose and other metrics that a user may want to monitor. The reports may convey a behavior modification recommendation based on the individualized metric. Behavior modification recommendations can include at least one of a type of food to eat, a sequence in which to eat different food types, a timing of meals during a day, a timing of exercise in relation to a meal or exercise. In step 670, at regular intervals recalibration of the entire system can be suggested with repeat glucose monitoring.

In some embodiments, other quantities can be measured in addition to glucose. For example, sensors for lactate, ketone, etc., can be utilized. These additional sensors can be separate sensors from the glucose sensor or can be combined into a single device with the glucose sensor to provide the metabolic data in step 640 to be used in the analyses. The additional sensors can help indicate further aspects of a person's metabolic response, such as during exercise. For example, higher ketone levels indicate more fat burning, and lactate levels indicate a shift between aerobic and anaerobic activity. Accordingly, additional metrics calculated and displayed to the patient may also include direct ratios of multiple metabolites such as glucose to ketone/lactate/free fatty acid or calculated metabolic indices such as glucose indices to ketone/lactate/free fatty acid indices. Correlations can be created between meal input ratios and these indices to generate individualized expert advice. In some embodiments, tracking of these additional aspects may be useful for athletes in determining a training program.

The meal parameters used in the analyses can include ratios of estimated carbohydrates, proteins, and fat content, as well as approximate caloric portion size. The system may request several mixed meals like a protein bar to sample a broad array and to provide better machine learning. These meals are then indexed along with metabolic sensor metrics and tracked (e.g., in a cloud-based infrastructure) for the individual patient.

Metabolic sensor data in step 640 may be augmented by additional sensor data such as heart rate, blood pressure, steps, weight, and/or accelerometer activity and sleep monitors, all of which may be transmitted to the mobile device, such as wirelessly. These measurements can be used to create correlations to the overall metrics as well. Aggregate data from the metabolic sensor (e.g., glucose, ketone, free fatty acid, etc.) and response to all activities (e.g., meals, sleep, exercise, general levels of activity) can have additional cross-correlations with heart rate, blood pressure, activity and other physical sensors included in the system and recorded in the databases.

FIG. 6B is a flowchart 680 showing a method of monitoring metabolic activity, in accordance with some embodiments. At step 682, a processor receives glucose data associated with an individual from a metabolic sensor. At step 684, the processor receives food intake information associated with the individual. At step 686, the processor calculates a plurality of global metrics. Each global metric is based on a glucose variability, a glucose load, and a post-prandial peak. The glucose variability is calculated from the glucose data associated with the individual. At step 688, the processor determines an individualized metric by correlating the food intake information associated with the individual to the plurality of global metrics. At step 690, the processor recommends a behavior modification based on the individualized metric.

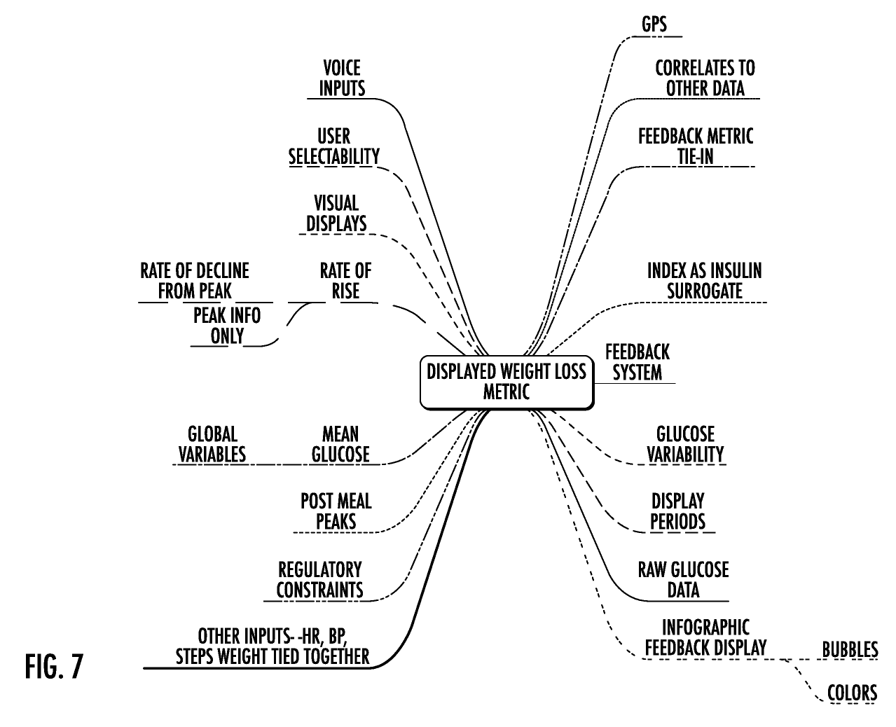

FIG. 7 is a tree diagram that depicts many of the inputs that could be used by artificial intelligence to perform the correlation and trend analysis to create tangible advice for the individual. For example, some embodiments involve using AI heuristics for creating success profiles for population cohorts. The success profiles may include success behaviors, food intake, exercise, and other factors in a proven weight loss cohort. Some embodiments may include using AI data summaries/correlates to be sent to an insurer, clinician, dietician, etc., for review, help with compliance and expert advice. AI may also be used to send user prompts or advice based on a success cohort, such as suggesting certain behavior based on an individual's index value.

Displays of meaningful indices/correlates, such as those in the reports of step 660, may be displayed in a simple, graphical format. As described earlier, each individual has different metrics and correlates based on analysis of their data. Weekly or monthly data can be aggregated and trended along with expert advice or reports that can be given via a patient caregiver or consultation. Displaying data or the behavior modification recommendation in a simple, graphical format keeps the user up-to-date in real-time so an immediate action can be performed based on the individualized metrics.

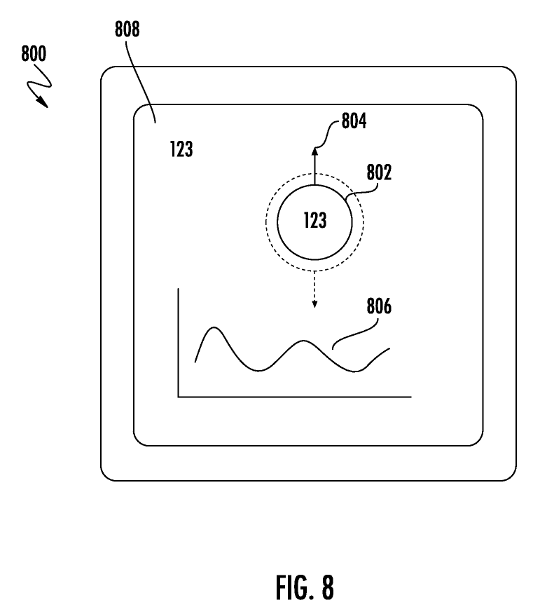

FIG. 8 shows an embodiment of a user interface 800 for the display of data. In this display screen user interface 800, a bubble style graphic 802 is utilized that increases in size and changes color with the increase of the metabolic index (i.e., global metric). Additionally, this embodiment shows the use of an up or down arrow 804 to indicate changes from prior states. The display screen could be an initial display of this bubble style graphic 802 to provide a quick view of the global metric. Also shown in FIG. 8 is a graph 806 of a moving index to indicate GV values over time, and another index 808 in the upper left corner which could be used, to show, for example, the number of calories. Other embodiments may include more in-depth analysis within layers of pull-down menus, and also feedback portions of the software application to provide directed advice. For example, the software application could include links to professionals such as a physician, dietician, or physical trainer.

FIG. 9 is another embodiment of a user interface 900 for the display of data. This embodiment has a bubble 902 showing the current glucose value 904, with the previous value 906 concentrically displayed in a contrasting format, such as a ghosted format. The bubbles 902 for the current value 904 and previous value 906 are sized to reflect their numerical values. The user interface 900 also displays a rate of change 908, embodied in FIG. 9 as a scale that may show a current value using, for example, highlighted numbers or a bar graph.

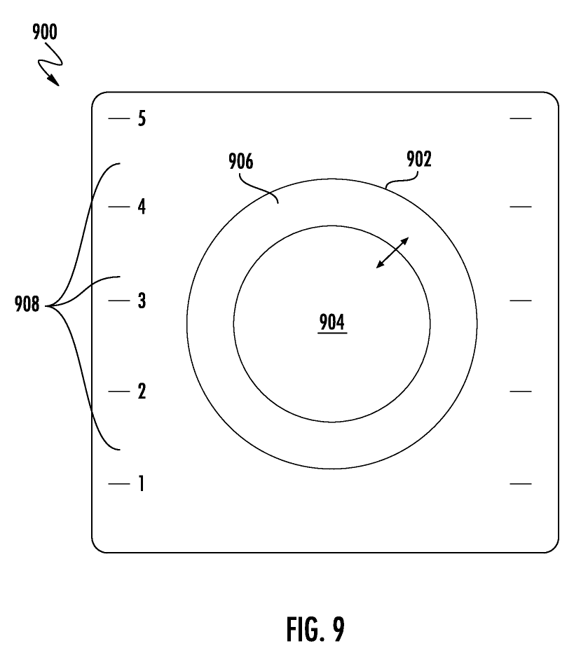

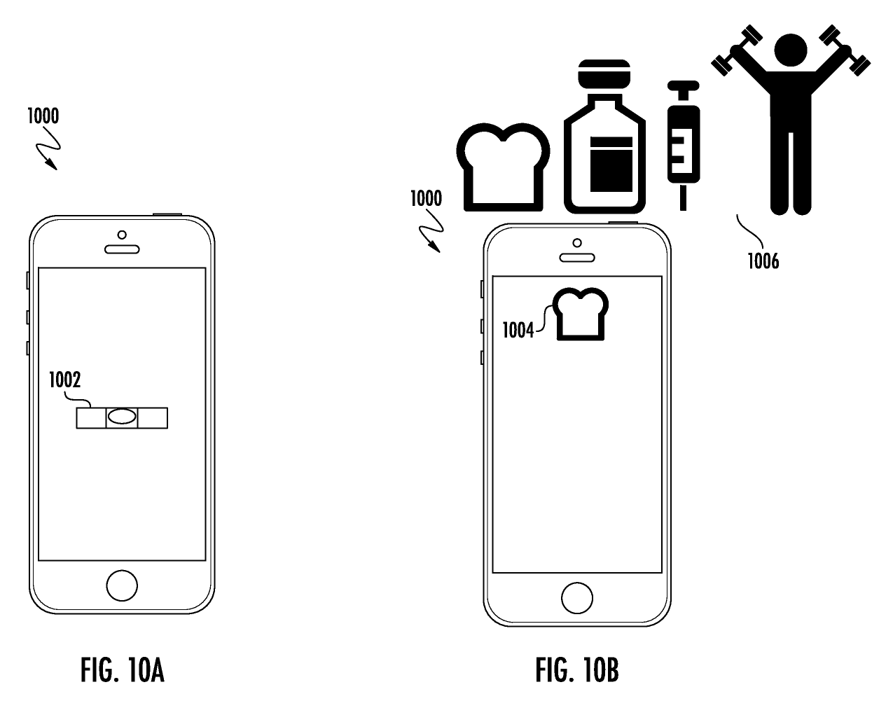

In some embodiments, the display of data may be a simple, visual cue for the user of the individualized metric such as the glucose level of the individual. FIGS. 10A and 10B are embodiments of a user interface 1000 for the display of data, in accordance with some embodiments. In FIG. 10A, a level 1002—a bubble in a liquid that shows adjustment to a horizontal by movement of the bubble relative to a central zone—is shown which indicates in the same manner as a typical handyman tool. Data from the CGM sensor 510 is transmitted to the device, and the user interface 1000 of the device visually indicates, for example, if the glucose value is high, low or within target by the level 1002. In this scenario as shown in FIG. 10A, the glucose value is level as indicated by the bubble between two vertical lines. However, if the glucose value is low, then the level 1002 would be displayed at an angle with the bubble to the left of the lower vertical line. In addition to the bubble in the level 1002 changing positions according to the glucose value, the level 1002 may change color such as green indicating an acceptable value, red indicating low and blue indicating high. In further embodiments, selecting the level 1002 when the glucose value is out of range, may launch a secondary display screen with recommendations of how to bring the glucose value back to within range (disclosed hereafter).

In FIG. 10B, the user interface 1000 depicts an icon 1004. The icon 1004 indicates an action to be taken. This may be helpful for the user not interested in numbers such as a child, or for international use, or any user who prefers a visual cue as a simple action to take when the glucose value is not within the acceptable range. For example, a slice of bread is shown which may be shown in different sizes to indicate “eat a snack” and how much to eat. A small icon 1004 of the slice of bread may be associated with a small snack. The icon 1004 may be chosen from a group of icons 1006 indicating to eat a snack, drink juice, take insulin or exercise. In other embodiments, more than one icon 1004 may be displayed at the same time depending on the data and what behavior modification is recommended.

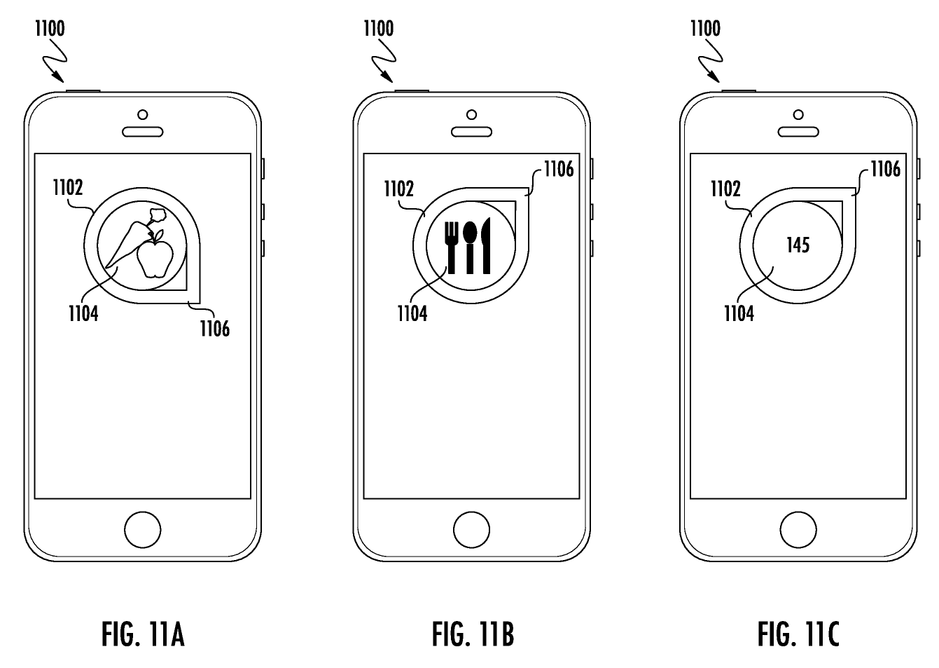

FIGS. 11A-11C illustrate user interfaces 1100 for the display of data, in accordance with some embodiments. The display shows a dial 1102 with an arrow 1106. With regard to the CGM sensor 510 data and the global metrics, the dial 1102 may display a picture 1104 of the recommendation while the arrow 1106 may indicate the blood glucose level as low, thus the arrow 1106 pointing downward (FIG. 11A) or as high, thus the arrow 1106 pointing upward (FIG. 11B). By clicking on or selecting the dial 1102, more data may be displayed such as the actual glucose value as shown in FIG. 11C, or another metric such as the amount of carbohydrates that corresponds to the recommendation such as an apple picture 1104 in the dial 1102.

There are benefits to the user by displaying metrics on the user interface in a simple format in real-time. This reduces the burden of use on the user because the user can quickly understand if an action needs to be performed to modify their glucose level. In some embodiments, the processor is part of a device having a display screen with a lock screen, home screen or wallpaper feature. The lock screen, home screen or wallpaper may be modified based on the glucose data associated with an individual from a metabolic sensor. In other words, the lock screen, home screen and/or wallpaper of the device may be changed based on the glucose data from the sensor. For example, when a high glucose level (high load or high variation metric) is detected, the screen may change to a yellow screen. When a normal glucose level (normal load and variation metric) is detected, the screen may change to a green screen. When a low glucose level (low load and high variation metric) is detected, the screen may change to a red screen. A metric such as the glucose level and/or the behavior modification recommendation such as an action may also be communicated.

The described embodiment is a fast, discreet, convenient method for the user to understand the metric by merely glancing at the device, such as the mobile phone, without opening a software application. Moreover, this is different than receiving a notification on the home screen of the mobile phone because the present embodiments work with the operating system of the mobile phone and change the image of the lock screen, home screen and/or wallpaper based on the sensor data monitoring the user without user input. This occurs automatically and in real-time. For example, the user may opt-in to this feature in the software application. The software application may trigger a “software flag” based upon the user's data via the sensor. The software flag is transmitted to interact with the operating system or home screen setting of the operating system, and the lock screen, home screen and/or wallpaper then changes color and/or displays an image based on the user settings. This may be similar to settings or software applications that change the lock screen, home screen and/or wallpaper based on time.

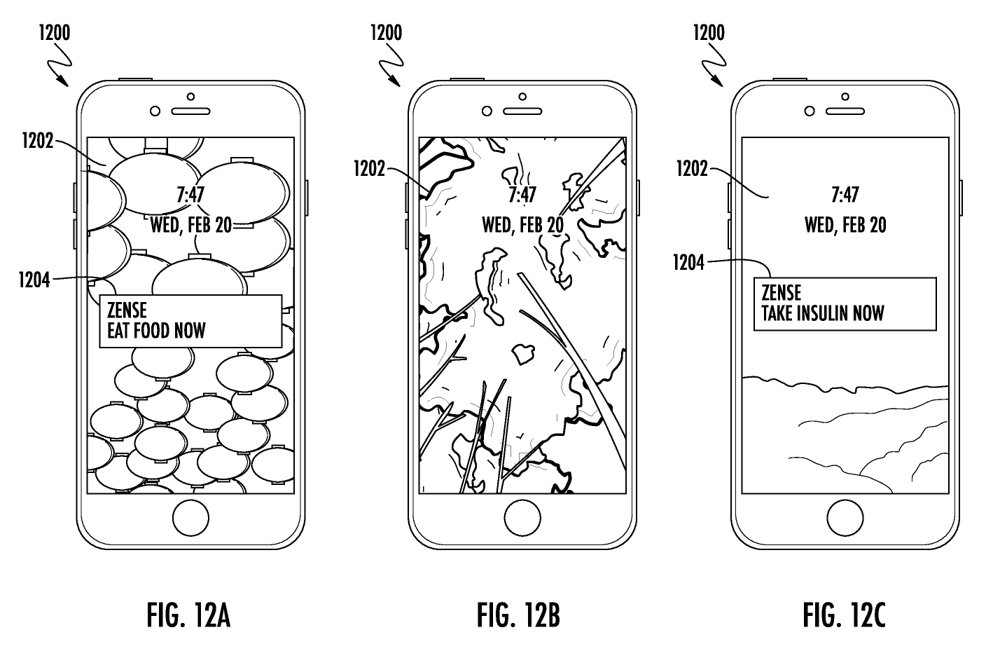

FIGS. 12A-12C depict embodiments of a user interface 1200 to communicate data, in accordance with some embodiments. The lock screen, home screen and/or wallpaper of the device, referred to as display screen 1202, may change color and/or display an image to indicate the glucose level. FIG. 12A illustrates the display screen 1202 with an image of red flowers or lanterns which may indicate low blood glucose levels, FIG. 12B illustrates the display screen 1202 as green leaves on trees which may indicate an in-range blood glucose level, and FIG. 12C illustrates the display screen 1202 as a yellow sunset which may indicate a high blood glucose level. The colors and/or images of the display screen may indicate other data such as another metric or an action. This is a discreet way to communicate the status of the user's health without other people in the vicinity knowing what the colors or images represent.

The behavior modification recommendation may be displayed in a banner 1204 on the display screen 1202 that is the lock screen, home screen and/or wallpaper of the device. For example, based on the individual data of the user, FIG. 12A illustrates the display screen 1202 with a red image indicating a low blood glucose levels and the banner 1204 with the action of “eat food now,” and FIG. 12C shows the display screen 1202 with a yellow image indicating a high blood glucose and the banner 1204 with the action as “take insulin now.”

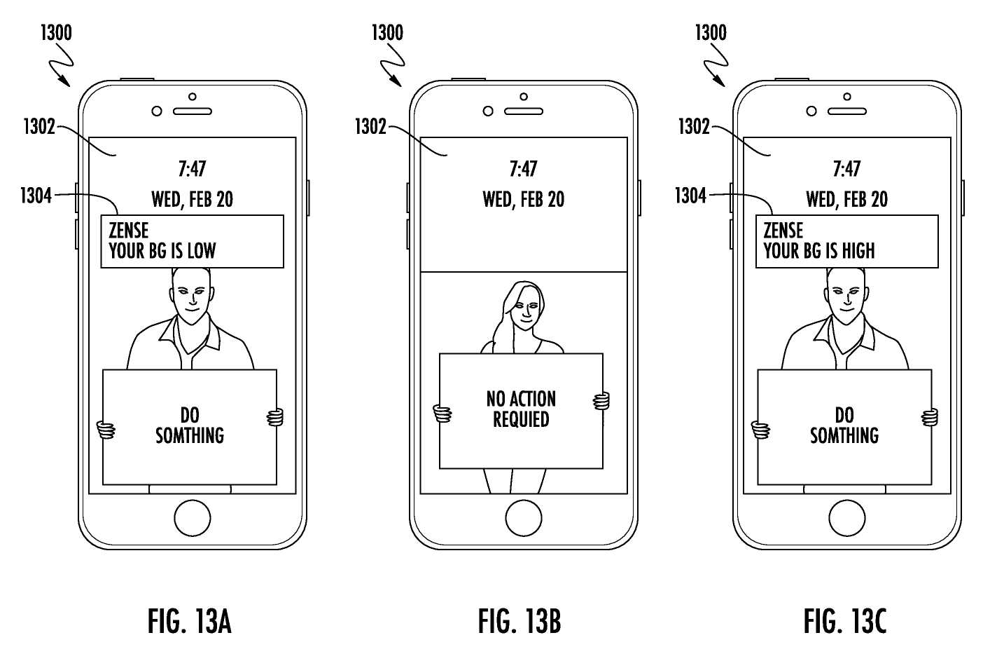

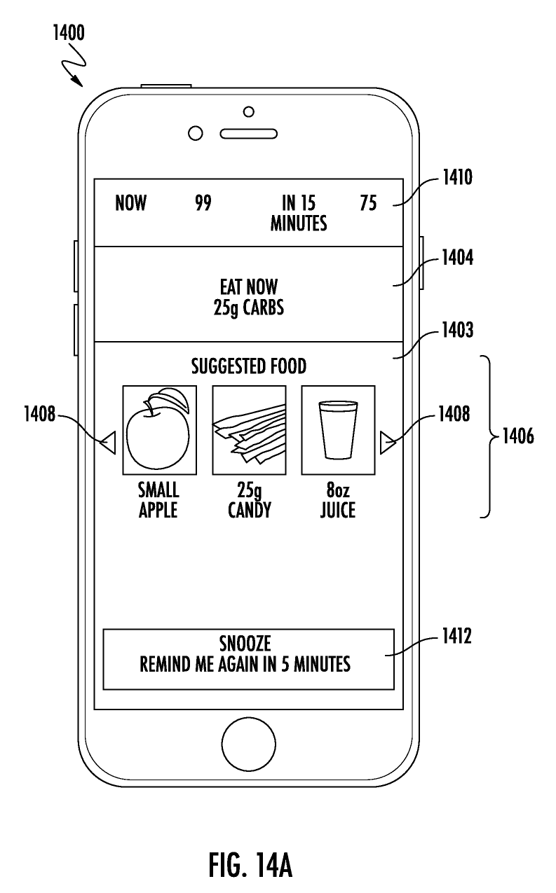

FIGS. 13A-13C depict other embodiments of a user interface 1300 to communicate data, in accordance with some embodiments. Similar to FIGS. 12A-12C, the display screen 1302 which is the lock screen, home screen and/or wallpaper of the device may change color and/or display an image to indicate the glucose level. In this scenario, only two different colors are used such as blue to indicate do something as in FIGS. 13A and 13C, and green to indicate the glucose level is in-range and no action is required. The banner 1304 on the display screen 1302 may indicate information about the metric such as the blood glucose is high (as shown in FIG. 13C) or the behavior modification recommendation such as to take medication (e.g., insulin) now.

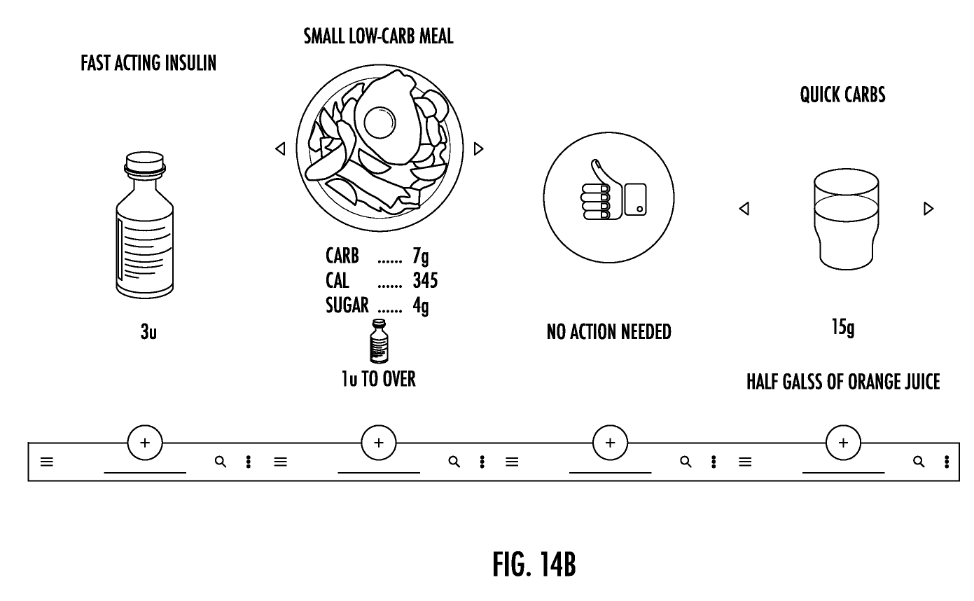

In some embodiments, more information or a deeper level of data may be needed to help guide the user. Referring to FIG. 12A, the user can click on the banner 1204 and a secondary user interface display screen opens. This may be an overlay on the lock screen, home screen and/or wallpaper, or a separate software application may open and the secondary screen is part of the software application. FIG. 14A shows an embodiment 1400 of a secondary user interface display screen 1403 to display data, in accordance with some embodiments. The banner 1404 repeats the action listed on banner 1204 and additionally, may recommend a specific amount or duration, e.g., in this case, grams of carbohydrates. This amount is what is needed in order to bring the user's blood glucose level back to into range. In section 1406, suggested foods are listed to achieve the recommended amount of carbohydrates. The suggested foods in section 1406 are not generic but specific candidates based on learning from historical food intake information received and historical metabolic index calculations. In some embodiments, the user can scroll through the suggested foods in section 1406 by clicking on arrows 1408. FIG. 14B shows examples of simple visuals of suggested foods, drinks or medications, in accordance with some embodiments, that may be shown in section 1406. Simple visuals with the number of units or carbohydrates listed may aid and train the user to understand what a specific amount of carbohydrates looks like.

A dashboard 1410 in FIG. 14A may list other metrics such as blood glucose stats for now and an estimate for 15 minutes later based on performing the recommendation such as eating a small apple. The estimate is also gleaned from historical food intake information received and historical metabolic index calculations since each individual has a different response to the same foods as demonstrated in FIG. 1. A footer 1412 may include a snooze function that can be selected by the user so that the user is reminded in a future amount of time such as 5 minutes. After the future amount of time, a visual or audio alert may be seen or heard.

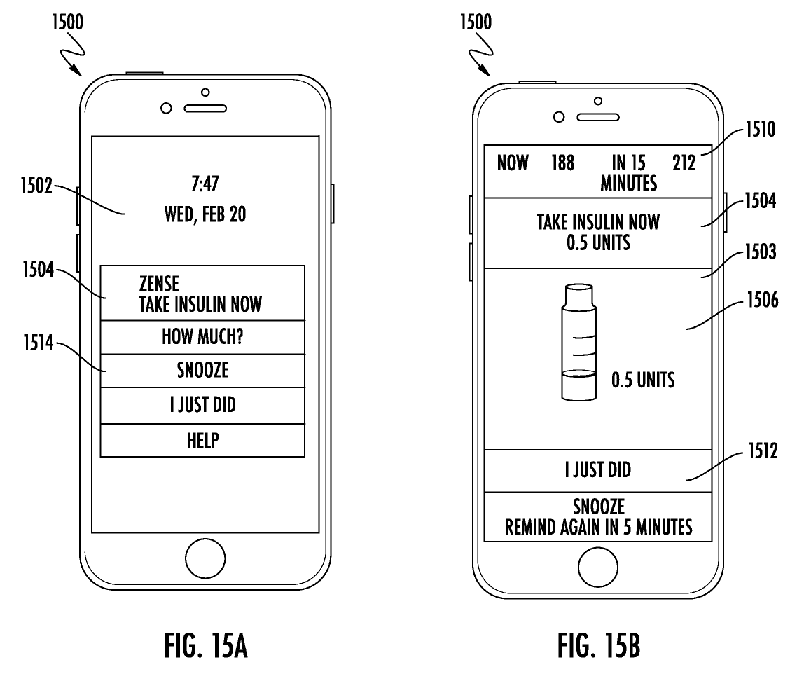

In another embodiment, FIG. 15A shows a user interface 1500 with a dropdown menu 1514 to further communicate data, in accordance with some embodiments. For example, if the user selects the banner 1504, the dropdown menu 1514 may appear as demonstrated in FIG. 15A. This may include another level of data such as a snooze feature on the display screen 1502 (as shown) or an amount or duration of the action displayed in the banner 1504. When the banner 1504 is selected again, the secondary user interface display screen 1503 opens. Screen 1503 is similar to the description of FIG. 14A. FIG. 15B is a secondary user interface display screen 1503 to display data, in accordance with some embodiments.

The banner 1504 repeats the action from the banner on the lock screen, home screen and/or wallpaper and additionally, recommends an amount of insulin to the consumer. This amount is what is needed in order to bring the user's blood glucose level back to in-range. In section 1506, a visual of the medication is shown. A dashboard 1510 may list other metrics such as blood glucose statistics for the present time and an estimate for in 15 minutes based on performing the recommendation such as consuming the insulin. A footer 1512 may include a snooze function that can be selected by the user so that the user is reminded in a future amount of time such as 5 minutes, or an option to confirm the action such as “I just did”. After the snooze amount of time, a visual or audio alert may be seen or heard.

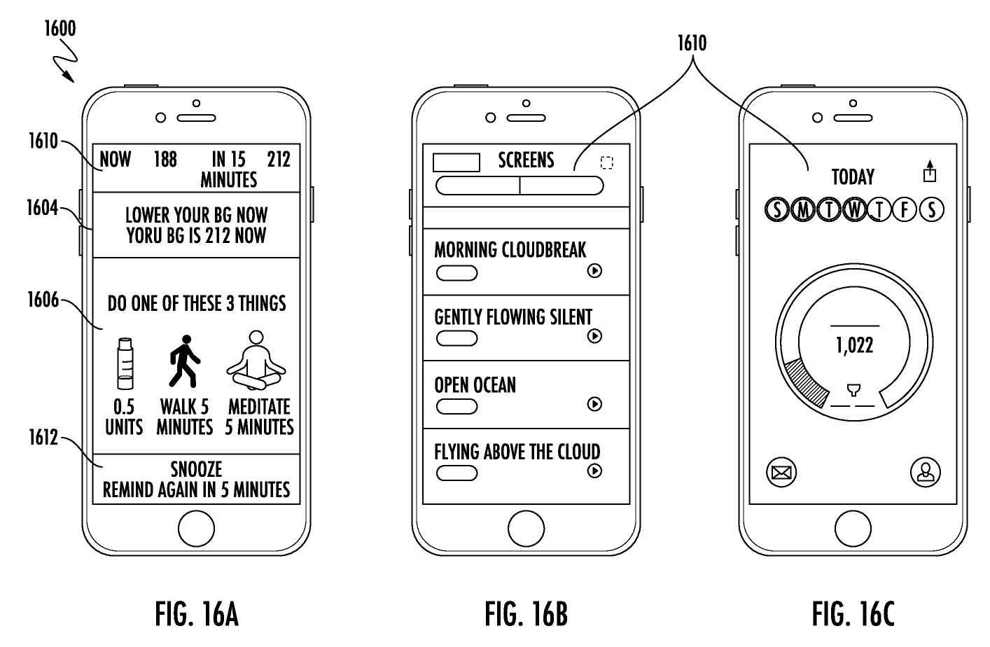

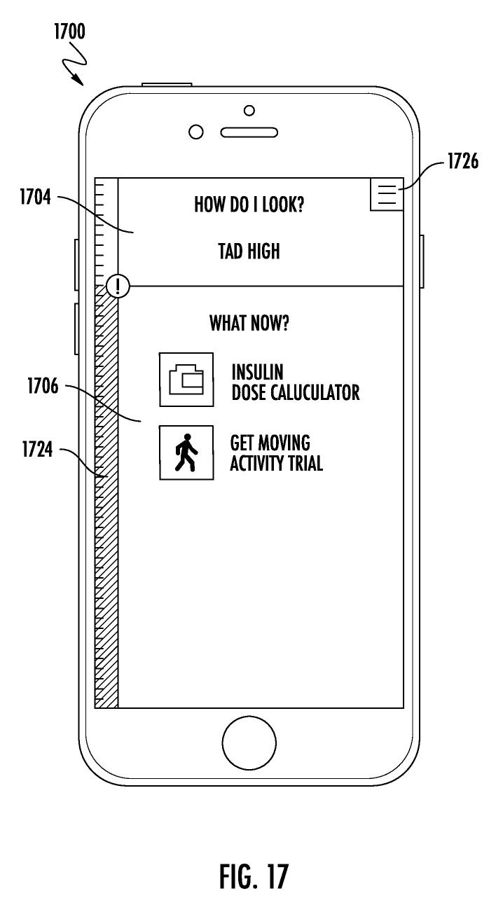

FIG. 16A is a user interface 1600 for the display of data, in accordance with some embodiments. The banner 1604, dashboard 1610 and footer 1612 are similar to the previous descriptions herein. In section 1606, suggested medication, activities or foods may be listed to achieve a blood glucose level within range. These are based on learning from historical food intake information received and historical metabolic index calculations for the specific individual. In some embodiments, by clicking on the visual in section 1606, a software application associated with the visual opens. In this way, when in the software application of the system, an unrelated-to-the-system second software application can be opened and accessed from the software application of the system. For example, by clicking on the meditation icon or the walk visual, an appropriate software application may open to track the data. These may be third-party software applications for exercise such as apps that track the daily number of steps taken per day. This data may be communicated to the system as feedback then used by the system for calculating and correlating metrics. FIGS. 16B and 16C depict example software applications in communication with the system, in accordance with some embodiments.

FIG. 17 is a user interface 1700 for the display of data, in accordance with some embodiments. The user interface 1700 may be on the lock screen, home screen and/or wallpaper, an overlay on the lock screen, home screen and/or wallpaper, or a separate software application. The banner 1704 communicates the status of a metric such as blood glucose. The section 1706 recommends the behavior modification such as medication, activities or foods to achieve a blood glucose level within range. These are based on learning from historical food intake information received and historical metabolic index calculations for the specific individual. A vertical bar indicator 1724 provides another visual cue of the metric. An options menu 1726 may include more in-depth analysis within layers of pull-down menus.

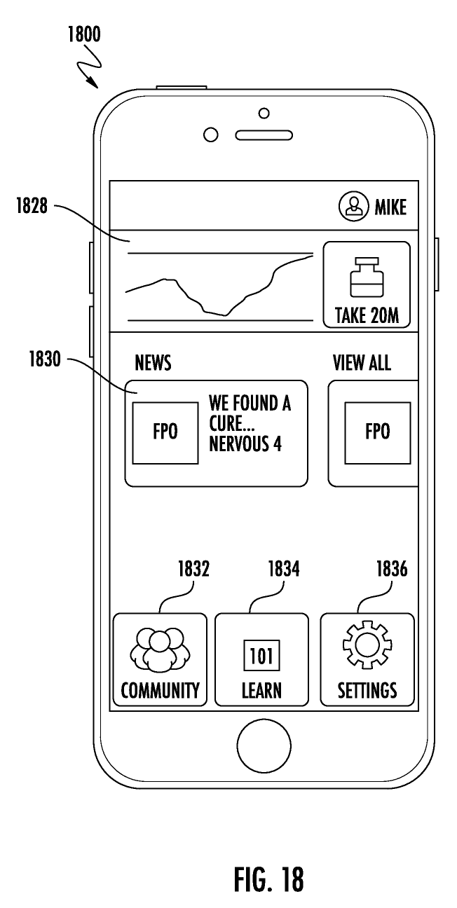

FIG. 18 is a user interface 1800 for the display of data, in accordance with some embodiments. As described with the various other example interfaces, the user interface 1800 may be on the lock screen, home screen and/or wallpaper, an overlay on the lock screen, home screen and/or wallpaper, or a separate software application. A status window 1828 communicates the status of a metric such as blood glucose as a graph over time without number labels. The graph terminates into a visual which is the behavior modification recommendation such as a medication, activities or foods to achieve a blood glucose level within range. The user interface 1800 may be more integrated with interests of the user by providing a media section with highlights and links to news articles 1830, community support 1832, training 1834 and settings 1836. By selecting the links of the media section, an unrelated-to-the-system second software application may open for the use of the user.

Some embodiments involve a system where understanding the individual's metabolic response to certain foods is used to guide the person to a metabolic data-driven weight loss program. For example, in some embodiments glucose (variation, total load and post-meal peaks) can be used for guiding weight loss. Further embodiments may use lactate sensing coincidently to distinguish exercise or other rises from food-related changes. Some embodiments may use a rate of decline in lactate levels from post-meal peaks to indicate fat storing.

Some embodiments may use global positioning system (GPS) data to provide supplemental information to the user. For example, based on an individual's GPS location, the system may provide locations of suggested restaurants and food recommendations served that the suggested restaurants. The system may also use location to encourage behavior modification, such as sending proactive texts or messages to not eat certain foods when the individual is identified as being in a location where that food is offered. GPS location data may also be used to map prior behaviors (e.g., food, others), or for “nagging” to prevent night binging. Another example of using GPS information in the present glucose monitoring and weight loss system includes using glucose peaks for retrospective understanding and behavioral monitoring.

Although embodiments have been described in relation to enhancing weight loss, the present glucose monitoring systems and methods can also be used to treat other diseases. For example, the present glucose monitoring systems and methods can be used for patients with cancer, from early stage to late stage, where the global metrics can be used to monitor food intake to reduce glucose variability. Glucose variability can uniquely serve as a proxy for insulin production and the presence of insulin-binding globulins and other potential growth-inducing factors that encourage cancer proliferation and increase the potential for recurrence or further metastases. In another example, the glucose monitoring can be used to address polycystic ovary syndrome, where modification of food intake can reduce glucose variability and insulin resistance, consequently improving fertility by increasing the chance of ovulation. Another example is the treatment of non-alcoholic fatty liver disease where glucose monitoring can prevent the elevation of glucose, which increases the deposition of fat in the liver that can then lead to, for instance, cirrhosis and liver failure.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. A method comprising:
receiving, by a processor, glucose data associated with an individual from a metabolic sensor;
receiving, by the processor, food intake information associated with the individual;
calculating, by the processor, a plurality of global metrics, wherein each global metric is based on a glucose variability, a glucose load, and a post-prandial peak, wherein the glucose variability is calculated from the glucose data associated with the individual;
determining, by the processor, an individualized metric by correlating the food intake information associated with the individual to the plurality of global metrics; and
recommending, by the processor, a behavior modification based on the individualized metric.

2. The method of claim 1, wherein the processor is in communication with or is part of a mobile device.

3. The method of claim 1, wherein the calculating of the plurality of global metrics comprises using weighting factors to combine the glucose variability, the glucose load and the post-prandial peak.

4. The method of claim 3, wherein the weighting factor comprises a derived function, the derived function being based on a weight category of the individual.

5. The method of claim 3, wherein the weighting factor is based on a rate of change of the post-prandial peak.

6. The method of claim 1, wherein receiving the food intake information comprises:
receiving an image of a food item;
using image recognition to identify the food item; and
receiving input on an amount of the food item consumed.

7. The method of claim 1, wherein receiving the food intake information associated with the individual comprises:
receiving an audio input of the food intake information; and
using voice recognition to analyze the audio input.

8. The method of claim 1, wherein the determining of the individualized metric comprises learning from historical food intake information received and historical metabolic index calculations.

9. The method of claim 1, wherein the behavior modification recommendation includes at least one of a type of food to eat, a sequence in which to eat different food types, a timing of meals during a day, a timing of exercise in relation to a meal and exercise.

10. The method of claim 1, wherein:
the processor is part of a device having a display screen with a lock screen, home screen or wallpaper feature;
the lock screen, home screen or wallpaper is modified based on the glucose data associated with the individual from the metabolic sensor.

11. A system comprising:
a) a metabolic sensor configured to measure glucose data associated with an individual; and
b) a processor configured to:
receive glucose data associated with the individual from the metabolic sensor;
receive food intake information associated with the individual;
calculate a plurality of global metrics, wherein each global metric is based on a glucose variability, a glucose load, and a post-prandial peak, wherein the glucose variability is calculated from the glucose data associated with the individual;
determine an individualized metric by correlating the food intake information associated with the individual to the plurality of global metrics; and
recommend a behavior modification based on the individualized metric.

12. The system of claim 11, wherein the processor is in communication with or is part of a mobile device.

13. The system of claim 11, wherein the processor calculates the plurality of global metrics by using weighting factors to combine the glucose variability, the glucose load and the post-prandial peak.

14. The system of claim 13, wherein the weighting factor comprises a derived function, the derived function being based on a weight category of the individual.

15. The system of claim 13, wherein the weighting factor is based on a rate of change of the post-prandial peak.

16. The system of claim 11, wherein the processor receives the food intake information associated with the individual by:
receiving an image of a food item;
using image recognition to identify the food item; and
receiving input on an amount of the food item consumed.

17. The system of claim 11, wherein the processor receives the food intake information associated with the individual by:
receiving an audio input of the food intake information; and
using voice recognition to analyze the audio input.

18. The system of claim 11, wherein the processor determines the individualized metric by learning from historical food intake information received and historical metabolic index calculations.

19. The system of claim 11, wherein the behavior modification recommendation includes at least one of a type of food to eat, a sequence in which to eat different food types, a timing of meals during a day, a timing of exercise in relation to a meal and exercise.

20. The system of claim 11, wherein:
the processor is part of a device having a display screen with a lock screen, home screen or wallpaper feature;
the lock screen, home screen or wallpaper is modified based on the glucose data associated with the individual from the metabolic sensor.
